(19) United States
(12) Patent Application Publication
(10) Pub. No.: US 2013/0150689 A1
(43) Pub. Date: Jun. 13, 2013

(54) DEVICE FOR SENSING A TARGET CHEMICAL AND METHOD OF ITS MAKING

(71) Applicant: Micropen Technologies Corporation, Honeoye Falls, NY (US)

(72) Inventor: Lori J. SHAW-KLEIN, Rochester, NY (US)

(73) Assignee: MICROPEN TECHNOLOGIES CORPORATION, Honeoye Falls, NY (US)

(21) Appl. No.: 13/707,255

(22) Filed: Dec. 6, 2012

Related U.S. Application Data
(60) Provisional application No. 61/569,035, filed on Dec. 9, 2011.

(51) Int. Cl. G01N 27/30; A61M 16/04; A61B 5/00; H04R 25/00; A61B 5/1473; A61B 5/1477

(52) U.S. Cl. CPC G01N 27/30; A61B 5/1473; A61B 5/1477; A61B 5/6852; A61B 5/6853; A61B 5/6862; A61B 5/686; A61B 5/6821; H04R 25/00; A61M 16/04; A61B 5/6811; A61B 5/6812
USPC 600/345; 204/400; 204/416; 204/403.14; 204/435; 427/2.11

(57) ABSTRACT

The present invention relates to a device for sensing a target chemical. The device includes a flexible, non-planar substrate; a printed, solid-state sensing element comprising a chemical sensing material which produces an electrical signal upon interaction with the target chemical; a first printed electrode comprising a first conductive composition; and a second electrode comprising a second conductive composition. The first and second electrodes are electrically isolated from one another, and one or both of the first and second electrodes is in electrical contact with said sensing element. The first and second electrodes and the sensing element collectively form an electrochemical sensor which is coupled to the flexible, non-planar substrate. Medical devices comprising the device of the present invention and methods of making a device for sensing a target chemical are also disclosed.

This application claims the benefit of U.S. Provisional Patent Application Ser. No. 61/569,035, filed Dec. 9, 2011, which is hereby incorporated by reference in its entirety.

### FIELD OF THE INVENTION

The present invention relates to a device for sensing a target chemical and a method of fabricating this device.

### BACKGROUND OF THE INVENTION

Often there is a need to detect the presence of and/or quantify the level of various species in biological systems in order to study and understand chemicals necessary for optimal biological function. Ideally, chemical sensors can be positioned on medical devices so that procedures and measurements may be simultaneously executed, and further invasive operations avoided. Chemical sensors can be based on colorimetric or optical responses. However, in those cases sensitive detectors must be included in close proximity to the sensor or, alternatively, the sensor must be removed from the biological environment in order to take a measurement. Electrochemical sensors provide a more attractive approach, yielding a response which can be transmitted electrically and thus be read directly in vivo.

Electrochemical sensors generally include a surface which is sensitive to the presence and concentration of ions, gases, or biological molecules, and responds to the presence of such a species by exhibiting a change in electrical properties. These electrical properties can be easily measured to detect or quantify the chemical species. The electrochemical sensors can be classified as amperometric, voltammetric, potentiometric, or conductometric depending on the mechanism and the mode of measurement of the electrochemical response. For example, ions in solution, or gas molecules, could interact directly with a metallic or inorganic oxide surface via a redox or catalytic reaction. Solid-state ion-selective electrochemical sensors are common and are often referred to as ion sensitive field effect transistors (“ISFET”). Such a surface could also be enzymatically modified in order to become directly sensitive to the presence of biological molecules. Similarly, molecules such as glucose or DNA can be selectively and specifically sensed through their interaction with such an enzyme.

Alternatively, an ionophore could be embedded in a polymeric matrix positioned over an electrode, forming a membrane-electrode structure which is sensitive to a specific ion. For example, valinomycin selectively enhances the diffusivity of potassium ions, while ionomycin is selective for calcium ions. β- and γ-cyclodextrins have demonstrated a selective response to promethazine, a histamine blocker. Such materials, for example, are commonly embedded in a layer of highly plasticized polyvinylchloride.

In medical applications, a particularly common and useful ion selective electrochemical sensor is the pH sensor, which is sensitive to the concentration of hydronium (H₃O⁻) ions in solution, which are formed by the protonation of water. For example, endoscopic capsules, such as the SmartPill®, detects changes in pH in order to identify physiological landmarks used in calculating regional transit times, as well as to indicate overall acid levels in the gastrointestinal system.

Another application for pH measurement is to monitor blood pH. Low blood pH may indicate, for example, respiratory depression, renal failure, or diabetes, while high blood pH may suggest over-ventilation. A solid state ion selective pH sensor, based on Al₂O₃ and positioned at the end of a polymeric catheter was proposed for this purpose by Cordis Corp. in the mid-1970's, as described by Bergveld et al., ISFET, Theory and Practice, IEEE Sensor Conference Toronto (2003).

Other medical applications for pH sensing have also been suggested, for example, in order to detect the excess hydrogen ions which can be formed at the surface of an implanted metallic stimulation electrode. If such an electrode is pulsed excessively, electrochemical reactions may occur resulting in local decreases in pH near the electrode, which in turn can damage surrounding tissue. An embedded pH sensor near such an electrode could present a convenient method to provide feedback and control stimulation signals.

Other ions important in biological function, such as potassium and sodium, may also be easily detected by ion selective electrochemical techniques. Such ions may be detected by sensors mounted in non-invasive devices in contact with blood, or have also been proposed to be incorporated in textiles in order to monitor such ions in sweat.

Pollutants and poisons may be detected electrochemically, and dissolved gases in biological liquid environments, including CO₂, NH₃, SO₂, NO and NO₂, may also be detected by careful selection of chemical sensing materials. Detection of such compounds is useful, for example, since high levels of nitrogen monoxide indicate that the asthma patient's air passages are about to become inflamed.

Glucose levels can be monitored by attaching a biosensor to an invasive medical device which contacts the blood, or by integrating a sensor into a contact lens, for instance, in order to measure the glucose level present in tears.

Such sensors can be manufactured on flat substrates by conventional means, including screen printing or vacuum deposition. However, if a sensor is desired on a non-planar substrate, it is made by methods which involve using a flat surface to affix the device after manufacture onto the non-planar substrate. This results in challenges in accurate positioning, good adhesion, conformality, and low surface roughness.

Electrochemical sensors have been incorporated on or in medical devices previously. For example, U.S. Pat. No. 4,981,470 to Bombeck, describes a pH sensor situated at the distal end of an endotracheal tube. The pH sensor is described as a commercially available sensor based on antimony, which must be attached to the end of the tube. Care must be taken that no sharp edges are exposed after attachment. Solvent treatment is suggested for creating a rounded surface.

U.S. Patent Application Publication No. 2010/0078030 to Colburn describes a carbon dioxide gas sensor positioned on an endotracheal tube, or similar airway device, positioned proximal to the inflatable cuff on the device. This sensor can provide information regarding the quality of the seal formed by the cuff against the tracheal walls, such that inflation pressure may be minimized to the point where the cuff is just functional, thereby reducing tissue damage. The carbon dioxide sensor may operate by any number of mechanisms, including optical, colorimetric, or electrochemical sensors. Electrochemical sensors may be screen printed. However, no indication is given as to how the sensors may be affixed to the medical device after manufacture.

U.S. Patent Application Publication No. 2010/0160756 to Petisce et al. describes multilayer bio sensors designed to be applied, for example, by screen printing, directly to a flexible substrate creating a flex circuit. This is then affixed to a medical device. This sequence also requires multiple steps and poses challenges for adhesion and smoothness of the final product.

U.S. Patent Application Publication No. 2010/0228110 to Tsoukalis describes glucose sensors, based on microfluidic constructs, within a rigid needle. Conductive paths leading to the electrodes can be produced by direct-write methods along the polymeric needle substrate.

U.S. Patent Application Publication No. 2010/0204554 to Say et al. describes analyte sensors for lactate, glucose, and oxygen situated on flexible substrates. Electrodes are conductive traces which may be made from conductive inks provided by various means including pad printing, inkjet printing, and similar technologies. However, the electrodes are deposited on flat substrates such as film, for which these printing techniques are optimal.

U.S. Patent Application Publication No. 2007/0270675 to Kane et al. describes an implantable medical device which includes a chemical sensor. Information on ions sensed is used to direct the device to administer appropriate treatment, such as an electrical pulse or delivery of a substance. Sensors may be deposited on the surface of the device through a variety of means including standard printing processes. However, the substrate printed is a planar film.

U.S. Pat. No. 7,534,330 to Yu et al. describes multilayer, miniaturized, implantable biosensors designed for blood analysis. The sensing element is comprised of an electrode which is coiled around an object, over which subsequent sensing and membrane layers are built up by solution deposition of the appropriate polymers and additives. While this approach allows for the application of a sensor on a curved surface, it requires winding and adhesion of the initial wire electrode, which is cumbersome.

The present invention is directed to overcoming these and other deficiencies in the art.

### SUMMARY OF THE INVENTION

One aspect of the present invention relates to a device for sensing a target chemical. The device includes a flexible, non-planar substrate; a printed, solid-state sensing element comprising a chemical sensing material which produces an electrical signal upon interaction with the target chemical; a first printed electrode comprising a first conductive composition; and a second electrode comprising a second conductive composition. The first and second electrodes are electrically isolated from one another, and one or both of the first and second electrodes is in electrical contact with said sensing element. The first and second electrodes and the sensing element collectively form an electrochemical sensor which is coupled to the flexible, non-planar substrate.

Another aspect of the present invention relates to a medical device comprising the device for sensing a target chemical of the present invention.

A further aspect of the present invention relates to a method of forming a device for sensing a target chemical. This method involves providing a flexible, non-planar substrate and printing an electrochemical sensor on said flexible, non-planar substrate. The electrochemical sensor comprises a first electrode comprising a first conductive composition and a solid-state sensing element comprising a chemical sensing material which produces an electrical signal upon interaction with the target chemical, wherein the sensing element is electrically coupled to the first electrode.

The present invention relates to electrochemical sensors formed on a flexible, non-planar substrate with a conductive composition. In one embodiment, the electrochemical sensors are formed by printing a conductive ink composition directly onto a flexible, non-planar substrate or surface of a medical device. More specifically, the present invention relates to sensors manufactured by direct writing technologies, and even more specifically, manufactured by precision syringe dispensing technologies such as Micropenning®. Such manufacturing methods are particularly unique in their capability to print on non-planar, flexible surfaces formed from, e.g., polymeric materials.

The present invention achieves its advantages by, e.g., avoiding the adhesion, attachment, and surface topographical issues described in U.S. Pat. No. 4,981,470 to Bombeck. In addition, the present invention may advantageously utilize procedures for direct write dispensing of ink compositions, including conductive ink compositions such as Micropen direct writing techniques. Conductive ink compositions are known to have excellent adhesion properties to substrates, without the need for costly or time consuming surface pretreatments. For a given substrate material, the ink composition may include a solvent which is capable of swelling or dissolving the substrate. Upon curing, the ink may leave behind a residue or a trace which is henceforth described as printed material. Furthermore, the ink may comprise a binder which is also capable of being dissolved in the solvent. The binder may be the same or different from the substrate polymer. However, to most accurately match the mechanical properties of the substrate material and the written trace, it may be desirable for the binder to be identical in composition to the substrate material.

According to the present invention, ink compositions are advantageously applied to, e.g., polymeric, flexible, non-planar substrates using any suitable printing technique to provide improved adhesion to substrates while maintaining the functional properties of the ink.

### BRIEF DESCRIPTION OF THE DRAWINGS

FIG. 1 is a perspective view of an electrochemical sensor on a non-planar substrate according to one embodiment of the present invention. The electrochemical sensor includes a sensing element, a working electrode, and a reference electrode, formed on a flexible, non-planar substrate.

FIG. 2 is a perspective view of an electrochemical sensor on a medical device that has a flexible, non-planar surface according to one embodiment of the present invention. The electrochemical sensor includes a sensing element, a working electrode, and a reference electrode, formed on at least a portion of a flexible, non-planar surface of an endotracheal tube.

FIGS. 3A-E are cross-sectional views of sequential fabrication steps in constructing a working electrode for an electrochemical sensor device according to one embodiment of the present invention. FIG. 3C is a cross-sectional view of first printed electrode 6 and sensing element 10 formed on flexible, non-planar substrate 4 of FIG. 1.

FIGS. 4A-C are cross-sectional views of sequential fabrication steps in constructing a reference electrode for an electrochemical sensor device according to one embodiment of the present invention. FIG. 4B is a cross-sectional view of second electrode 8 formed on flexible, non-planar substrate 4 of FIG. 1.

FIG. 5 is a cross-sectional view of a first printed electrode formed on the endotracheal tube of FIG. 2.

### DETAILED DESCRIPTION OF THE INVENTION

A first aspect of the present invention relates to a device for sensing a target chemical. The device includes a flexible, non-planar substrate; a printed, solid-state sensing element comprising a chemical sensing material which produces an electrical signal upon interaction with the target chemical; a first printed electrode comprising a first conductive composition; and a second electrode comprising a second conductive composition. The first and second electrodes are electrically isolated from one another, and one or both of the first and second electrodes is in electrical contact with said sensing element. The first and second electrodes and the sensing element collectively form an electrochemical sensor which is coupled to the flexible, non-planar substrate.

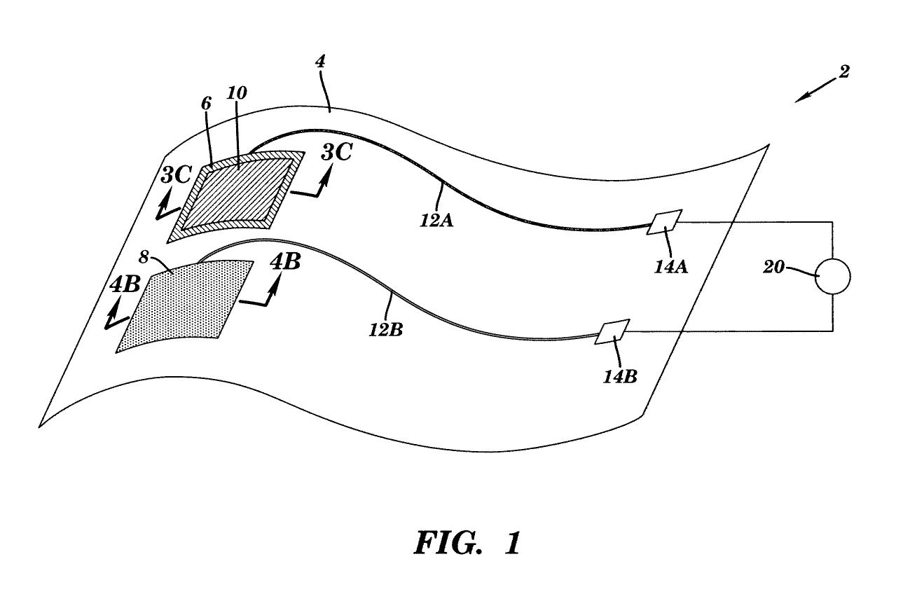

With reference to FIG. 1, device 2 for sensing a target chemical has flexible, non-planar substrate 4, upon which is formed first printed electrode 6 and second electrode 8. In the particular embodiment illustrated in FIG. 1, solid-state sensing element 10 is in electrical contact with first printed electrode 6 by being positioned on top of a portion of first printed electrode 6. Conductive trace 12A extends from first printed electrode 6 to conductive pad 14A. Conductive trace 12B extends from second electrode 8 to conductive pad 14B. First printed electrode 6, second electrode 8, and sensing element 10 collectively form what is referred to herein as an electrochemical sensor. In the particular embodiment illustrated in FIG. 1, electrical measurement device 20 is connected to both conductive pad 14A and conductive pad 14B.

As illustrated in FIG. 1, substrate 4 upon which first printed electrode 6 and second electrode 8 are formed is a flexible, non-planar substrate. Non-planar substrate 4 may have a regular or smooth surface, or an irregular or rough surface. Substrate 4 may be constructed of any material capable of receiving a conductive ink composition including, without limitation, polymeric materials known in the art such as polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polycaprolactone, and copolymers or mixtures thereof. Many such materials are commonly known to be used in fabricating medical devices and instrumentation.

Referring still to FIG. 1, according to one embodiment of the present invention, first printed electrode 6, second electrode 8, conductive traces 12A and 12B, and conductive pads 14A and 14B are formed from conductive inks Suitable conductive ink compositions include those described in U.S. Patent Application Publication No. 2010/0119789 to Grande, which is hereby incorporated by reference in its entirety. Conductive ink compositions may include various metal or metal-containing materials, e.g., copper, silver, gold, palladium, platinum, nickel. Suitable conductive ink compositions may also include various forms of conductive carbon (e.g., graphite, carbon black, carbon nanotubes), conductive ceramics (e.g., tin oxide, vanadium pentoxide, doped versions of the tin oxide, or doped versions of vanadium oxide), conducting polymers (e.g., polypyrrole, polythiophene, or polyaniline), and/or combinations thereof. Conductive ink compositions may also include various combinations, mixtures, or copolymers of the above mentioned materials. One or more polymers may be present to bind, e.g., conductive particles together and to provide enhanced adhesion to the substrate upon which the conductive ink is deposited. One or more solvents or carriers may also be present in the ink to dissolve or disperse the components of the ink, and/or to provide interaction with the substrate, thereby enhancing adhesion. Additional additives may include surfactants, thickeners, dispersants, defoamers, and the like. Surfactants, defoamers, or dispersants may be present to facilitate or inhibit spreading on the substrate, improve handling of the ink, improve the quality of the dispersion, or change the coefficient of friction of the dried ink. Particles may be introduced to tune ink rheology or to introduce roughness or porosity to the polymeric material's interior or exterior surface. The ink composition can also comprise one or more surface active agents, rheology modifiers, lubricants, matting agents, or spacers. The conductive compositions may further include other additives commonly used in ink compositions.

Conductive ink compositions of the present invention may include a binder. Suitable binders may include, without limitation, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polyethylene, polytetrafluoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polyisoprene, polycaprolactone, cyanoacrylates, polyvinyl butyral, polyvinyl formal, polyethylene oxide, polyvinyl alcohol, polyvinylpyrrolidone, cellulose esters, cellulose ethers, carrageenan, gelatin, chitosan, and mixtures or copolymers thereof.

The thickness of each conductive structure (e.g., printed material forming the electrochemical sensor) on the substrate may be controlled or modified depending on the particular method of forming the structure (described in more detail infra). In one embodiment, the conductive structure is from 1 to 500 microns thick. In another embodiment, the conductive structure is from 20 to 100 microns thick. In yet another embodiment, the conductive structure is from 30 to 80 microns thick.

With further reference to FIG. 1, sensing element 10 is a solid-state sensing element and, in one embodiment, is made wholly or at least partially of a chemical sensing material which produces an electrical signal when it interacts with a target chemical. This chemical sensing material may be, for example, an ionophore, an enzyme, a macromolecule, a metal, a metal oxide or a metal nitride, or an insertion compound (such as cyclodextrin, zeolite, or other material which physically entraps target species through geometrical action), or combinations thereof.

As with first printed electrode 6, second electrode 8, conductive traces 12A and 12B, and conductive pads 14A and 14B, sensing element 10 may be formed as a conductive ink applied to a substrate (or surface) of device 2. The thickness of sensing element 10 may vary. In one embodiment, sensing element 10 is from 1 to 500 microns thick. In another embodiment, sensing element 10 is from 20 to 100 microns thick. In yet another embodiment, sensing element 10 is from 30 to microns thick.

According to one embodiment, an ionophore is embedded in a polymeric matrix and is positioned over (i.e., on top of) at least a portion of, e.g., first printed electrode 6. This effectively forms a membrane-electrode structure which is sensitive to a specific ion. For example, valinomycin selectively enhances the diffusivity of potassium ions, while ionomycin is selective for calcium ions and nonactin specifically interacts with ammonium. β- and γ-cyclodextrins have demonstrated a selective response to promethazine, a histamine blocker. Such materials are, for example, commonly embedded in a layer of highly plasticized polyvinylchloride.

In general, enzyme field effect transistors (“FETs”) are based on the principle of pH-sensitive ISFETs in which the concentration of hydrogen ions during an enzymatic reaction is proportional to the level of a sensed substance. The enzyme can be chemically bound to the electrode surface or added to a membrane formed over the electrode.

Classes of useful enzymes include, but are not limited to, esterases, hydrolases, oxidoreductases, peroxidases, luciferase, kinases, lipases, phosphatases, proteases, and oxidases. Specific examples (and what they are sensitive to) include glucose oxidase (glucose), glucose dehydrogenase (glucose), alcohol dehydrogenase (primary alcohols), horseradish peroxidase or bromoperoxidase (H₂O₂), cholesterol oxidase and cholesterol esterase (cholesterol), choline oxidase and phospholipidase D (choline phospholipids), lactate oxidase (lactose), sarcosine oxidase and creatinase (creatine and creatinine), glutamate deydrogenase (ammonia), lactate oxidase (lactate), uricase (uric acid), or acetylcholinesterase (arsenic). Peroxidase coupled with a specific mediator (3,3′,5,5′-tetramethybenzidine) has also been used in a solid-state sensor for DNA detection.

Other molecules, which are not specifically ionophores or enzymes but are suitable for forming a solid-state sensing element of the present invention, may selectively interact with biological molecules of interest, such that they too influence the mobility of associated ions resulting in an electrochemical signal. For example, thiol-modified oligonucleotides have demonstrated specificity for DNA sensing, while protamine sulfate can be used for sensing heparin levels. Conductive polymers can also be altered by direct absorption of species of interest and used as conductimetric electrochemical sensors. For example, polyaniline and polypyrrole have both been used for pH measurements via electrochemical interactions.

Noble metals such as silver, palladium, gold, and the like can be used as potentiometric sensors for the detection of anions such as Cl⁻, I⁻ and Br⁻, as well as other species. However, metal oxides or nitrides are the most common surfaces used to sense ions. For example, pH sensors may include, but are not limited to, IrO₂, RuO₂, Si₃N₄, Al₂O₃, Ta₂O₅, SnO₂, PbO₂, TiO₂, WO₃, MnO₂, RhO_{2, OsO}₂, PdO, ZrO₂, Y₂O₃-stabilized ZrO₂, AlN, GaN, and the like.

Ammonia can be effectively detected by MoO₃, Bi₂O₃, V₂O₅; while NOₓ detection has been demonstrated with indium tin oxide or yttria-stabilized zirconia surfaces. N₂ has been detected due to its interaction with a surface of LaFeO₃, while CO₂ has been detected on SmFeO₃. SnO₂ has been used in CO electrochemical detectors, while MgAl₂O₄ can detect H₂O vapor levels. O₂ detection has been reported on TiO₂, SrTiO₃, BaTiO₃, ZrO₂, Fe₂O₃, CoO, ZnO, SnO₂, and La₂O₃. H₂ detection has been successfully demonstrated using Co₃O₄, ZnO, SnO₂, MoO₃, WO₃, and MnO₂. Niobium and platinum-doped TiO₂ has been used for ethanol and acetone sensing.

In the event that the chemical sensing material of the sensing element is sensitive (i.e., to contact or exposure), an overcoat layer or a membrane may be provided over at least a portion of the chemical sensing material to limit the diffusion of unwanted species, but to still allow species of interest to pass freely through. Overcoat layers, membranes, and/or adhesion promoting layers or treatments may be present as required to gain adequate mechanical or functional properties. Many polymeric materials known in the art can be used to make such layers. Suitable materials may include, for example, epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), ionomers, and mixtures or copolymers thereof. A non-limiting example of a common membrane material for solid state applications is Nafion® sulfonated perfluorinated ionomer.

The device of the present invention may optionally include an intermediate layer positioned between the substrate and any of the first printed electrode, the second electrode, the sensing element, the conductive traces, and the conductive pads. A suitable intermediate layer may be formed, for example, from epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), and mixtures or copolymers thereof.

While the particular embodiment illustrated in FIG. 1 shows a single sensing element 10 on device 2, the present invention contemplates the use of more than one, and even several sensors positioned in e.g., an array on a surface in order to simultaneously sense several materials or compounds at once. Thus, the device for sensing a target chemical according to the present invention need not be limited to one sensing element or electrochemical sensor (formed from the first and second electrodes and a sensing element), but can comprise a plurality of sensing elements or electrochemical sensors. According to this embodiment, the device for sensing a target chemical may function, e.g., as an “electronic nose” or “electronic tongue.”

In one embodiment, the device of the present invention includes at least one reference electrode comprising a conductive composition proximate to or on the substrate of the device. Thus, for example, with reference to FIG. 1, second electrode 8 may be a reference electrode which preferably does not react with the target chemical to be sensed. Alternatively, second electrode 8 is a reference electrode that reacts in a completely understood and predictable manner, thereby providing a comparative position against which to measure electrical changes resulting from the presence of the target chemical on a working electrode (e.g., first printed electrode 6). The reference electrode may or may not be electrically connected to the sensing element. In solid-state sensors, a commonly used and well-characterized material for forming a reference electrode is silver-silver chloride. Several commercial screen-printing inks are available with such compositions. Other combinations or materials such as graphite-silver chloride and IrOₓ have also been used with some success. As for working electrodes, the reference electrode may be at least partially covered with a membrane (as discussed in more detail below), thereby limiting the diffusion of unwanted species.

Auxiliary or counter electrodes may also be present on the device of the present invention, especially in voltammetric electrochemical sensing. When employed, auxiliary or counter electrodes may be formed from, e.g., a noble metal such as platinum, to serve as an electrical conductor from the source through the solution to the microelectrode. The basic requirement of a counter electrode is to provide an alternative route for the current to follow, so that only a small current flows through the reference electrode.

Device 2 of FIG. 1 includes electrical measurement device 20 connected to conductive traces 12A and 12B at, in the embodiment shown in FIG. 1, conductive pads 14A and 14B. Electrical measurement device 20 may be any of a variety of devices capable of taking electrical measurements as received from sensing element 10 and communicated through the electrochemical sensor by means of first printed electrode 6 and second electrode 8 being electrically isolated from one another. Specific non-limiting examples of electrical measurement devices include a voltmeter, ohmmeter, oscilloscope, and ammeter.

In one embodiment of the present invention, the device for sensing a target chemical of the present invention is included on a medical device. Thus, another aspect of the present invention relates to a medical device comprising the device for sensing a target chemical of the present invention. Examples of medical devices suitable for containing an electrochemical sensor device of the present invention include, without limitation, endotracheal tubes, endobronchial tubes, cannulae, catheters, balloons, stents, airways, sensors, stimulators, implants, intraocular or contact lenses, cochlear implants, and orthopedic implants or prostheses.

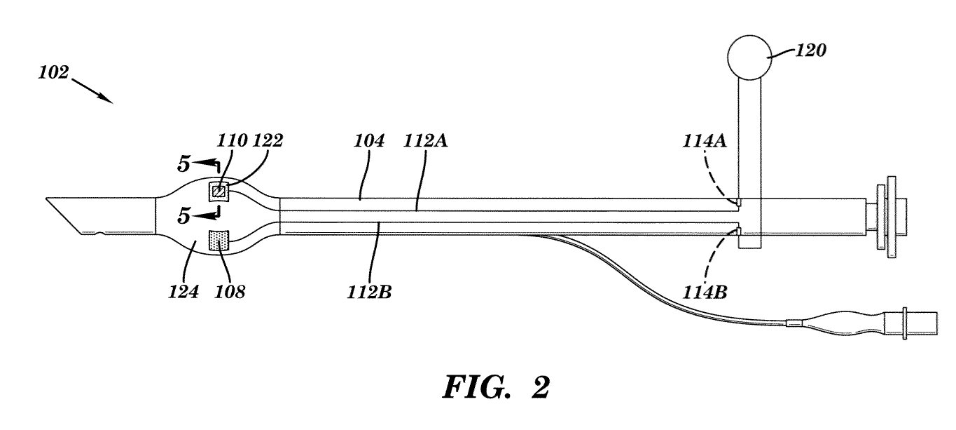

FIG. 2 illustrates one embodiment of a medical device having the device for sensing a target chemical of the present invention printed on the surface thereof. As illustrated in FIG. 2, device 102 is an endotracheal tube that includes flexible, non-planar substrate (or surface) 104, upon which is formed a first printed electrode and second electrode 108. The first printed electrode is not shown in FIG. 2, because it is covered by overcoat layer 122 and sensing element 110. (A cross-section of first printed electrode 106, substrate 104, overcoat layer 122, and sensing element 110 is illustrated in FIG. 5.) In the particular embodiment illustrated in FIG. 2, sensing element 110 is in electrical contact with the first printed electrode by being positioned on top of a portion of the first printed electrode. Conductive trace 112A extends from the first printed electrode (beneath overcoat layer 122 and sensing element 110) to conductive pad 114A. Conductive trace 112B extends from second electrode 108 to conductive pad 114B. In the particular embodiment illustrated in FIG. 2, electrical measurement device 120 is connected to both conductive pad 114A and conductive pad 114B.

As illustrated in FIG. 2, the first printed electrode (not shown in FIG. 2 because it resides beneath overcoat layer 122 and sensing element 110), second electrode 108, sensing element 110, overcoat layer 122, and a portion of conductive traces 112A and 112B are formed on flexible, non-planar inflated cuff 124.

Another aspect of the present invention relates to a method of forming a device for sensing a target chemical. This method involves providing a flexible, non-planar substrate and printing an electrochemical sensor on said flexible, non-planar substrate. The electrochemical sensor comprises a first electrode comprising a first conductive composition and a solid-state sensing element comprising a chemical sensing material which produces an electrical signal upon interaction with the target chemical, wherein the sensing element is electrically coupled to the first electrode.

According to one embodiment of the method of the present invention, the electrochemical sensor further includes a second electrode electrically isolated from the first electrode, the second electrode comprising a second conductive composition.

In one embodiment, printing the electrochemical sensor is carried out by direct writing techniques. According to this embodiment, one or more of the first electrode, the second electrode, conductive traces, and conductive pads are formed from a conductive ink using a Micropen (Micropen Technologies Corp., Honeoye Falls, N.Y. or NScrypt® technologies). Such techniques are well described in Pique et al., Direct-Write Technologies for Rapid Prototyping Applications: Sensors, Electronics, and Integrated Power Sources, Academic Press (2002), which is hereby incorporated by reference in its entirety. Direct writing techniques have been disclosed as methods for applying surface layers such as drug-eluting layers for stents (U.S. Patent Application Publication No. 2008/0071352 to Weber et al., which is hereby incorporated by reference in its entirety). These approaches can be modified such that they would be applicable to methods of the present invention.

Direct writing techniques, such as Micropenning®, are particularly preferred for making devices of the present invention due to their ability to accommodate inks having an extremely wide range of rheological properties and very high solids levels. Micropenning® also has excellent three dimensional substrate manipulation capabilities. In the present invention, an ink displacement pen can be used to apply or deposit the electrochemical sensor in any design or pattern, including interconnected or layered structures. This technique accommodates a wide range of ink viscosity, so that any material which can be successfully dissolved or dispersed in a liquid and forms a continuous layer or marking when dry, can be formed into a polymeric material which is adhered to the surface of the device. Furthermore, the disadvantages of laser machining, including burr formation, sharp edges, inadvertent heating, and material waste are not a concern with such deposition techniques.

Following application of a conductive composition material the substrate, the conductive composition may be cured. Curing methods are well known in the art. Methods such as baking, radiant heat, UV or IR irradiation, aeration, or letting the substrate stand in air so that the solvent in the ink is evaporated can be used to cure the conductive compositions and convert them to a printed material which is adhered to the substrate (or surface).

In one embodiment, the device for sensing a target chemical of the present invention is such that one or more of the first electrode, the second electrode, the conductive traces, and the conductive pads has at least one layer. If any of these structures has multiple layers, the layers could be deposited on top of each other such that they are joined together at their surfaces. They can be made of the same material or different materials. According to one embodiment, the printed structure comprises at least two printed materials. For example, the first deposition or layer can be a first color, function, or composition, and the second deposition or layer can be a second color, function, or composition.

An advantage of using a conductive ink to form, e.g., one or more of a first electrode, a second electrode, conductive traces, and conductive pads is that the conductive ink can be deposited on a variety of flexible, non-planar substrates because conductive inks can be made to have, and indeed often have, enhanced adhesion to substrates.

In the method of the present invention, formation of the electrochemical sensor may involve the use of a solvent. Suitable solvents include, without limitation, solvents based on a paraffinic hydrocarbon, an aromatic hydrocarbon, a halohydrocarbon, an ether, a ketone, an aldehyde, an ester, a nitrogen-containing solvent, a sulfur containing solvent, an alcohol, a polyhydric alcohol, a phenol, water, and mixtures thereof.

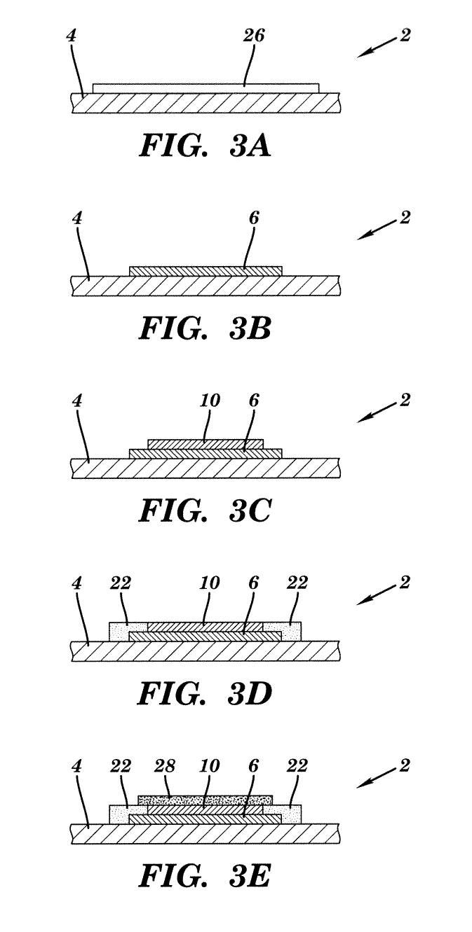

FIGS. 3A-E illustrate some of the steps carried out to perform the method of the present invention. In particular, FIGS. 3A-E are cross-sectional views of sensing element 10 and first electrode 6 formed on substrate 4 (see FIG. 1). In FIG. 3A, intermediate layer 26 is formed onto substrate 4. Intermediate layer 26 is optional, and may be used to assist in forming other structures of the electrochemical sensor of the present invention. For example, intermediate layer 26 may be used to improve adhesion between substrate 4 and the various components of the electrochemical sensor formed thereon. Intermediate layer 26 may also be employed to provide additional stiffening of device 2 if device 2 or substrate 4 proves too flexible to adequately support the electrochemical sensor structure. Alternatively, intermediate layer 26 may be used to provide electric isolation if device 2 or substrate (or surface) 4 possess an electronic or ionic conductivity such that it interferes with the proper functioning of first electrode 6. Note that for simplicity of presentation, the presence of intermediate layer 26 is omitted in FIGS. 3B-E.

In FIG. 3B, first electrode 6 is formed onto substrate 4. In FIG. 3C, sensing element 10 is applied on top of a portion of first electrode 6, which resides on substrate 4. In FIG. 3D, overcoat layer 22 is formed on the portion of first printed electrode 6 that is not covered by sensing element 10 and a portion of substrate 4. As illustrated, overcoat layer 22 is used to encapsulate edges of first electrode 6. Overcoat layer 22 protects first electrode 6 from, e.g., ions, moisture, and/or friction and may provide support against stress. In addition, overcoat layer 22 may be used as a means of enhancing flexibility and providing support to device 2. Further, overcoat layer 22 may contain additives that import desirable properties such as radio opacity, or release of medicaments or other substances. Overcoat layer 22 could also be used to ensure that any irritation or toxicity inherent to the material used to form first printed electrode 6 or its binder is isolated from, e.g., body tissues. Any biocompatible, non-conductive, impermeable polymer which is easily applied may be used in overcoat layer 22. In FIG. 3E, membrane 28 is formed on top of sensing element 10 and a portion of overcoat layer 22. Overcoat layer 22 is formed on the portion of first printed electrode 6 that is not covered by sensing element 10 and a portion of substrate 4. Membrane 28 may provide selective transport of particular species to sensing element 10 and a barrier to other species present which might add to or interfere with the signal generated by a target chemical.

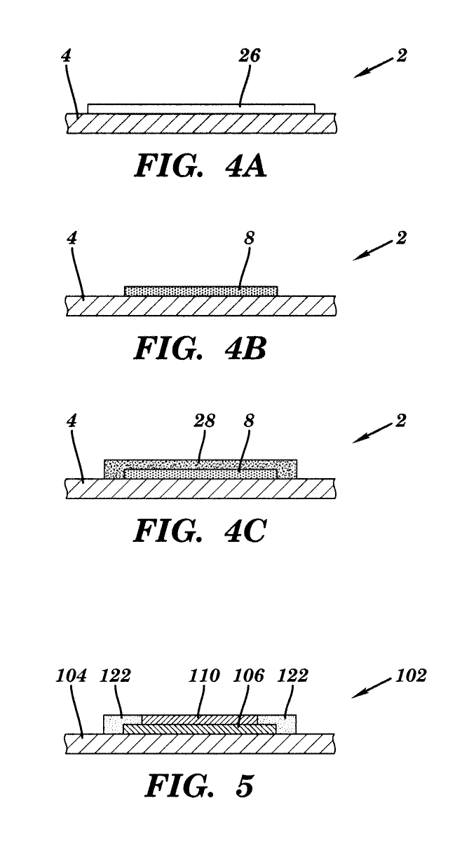

Turning now to FIGS. 4A-C, additional steps of carrying out the method of the present invention are shown. In particular, FIGS. 4A-C are cross-sectional views of second electrode 8 (see FIG. 1). In FIG. 4A, intermediate layer 26 is formed onto substrate (or surface) 4. Note that for simplicity of presentation the presence of intermediate layer 26 is omitted in FIGS. 4B-C. In FIG. 4B, second electrode 8 is provided directly on substrate (or surface) 4. In FIG. 4C, membrane 28 is disposed over second electrode 8, which resides on substrate (or surface) 4.

### EXAMPLES

The following examples are provided to illustrate embodiments of the present invention but are by no means intended to limit its scope.

### Example 1

### Measurement of Hydrogen Ion Concentration

For production of silver electrodes, polyvinyl chloride (Aldrich, high molecular weight) was dissolved in cyclohexanone at a concentration of 12% by weight, and silver flakes (Ames-Goldsmith, MBT-79) were added to bring the ratio of silver to polymer to 92:8. The total solids present in the ink composition were 63% by weight. The materials were dispersed and deaerated using a centrifugal planetary mixer (Kurabo Mazerustar model KK-50S).

The ink was loaded into a syringe and extruded through a Micropen dispensing apparatus onto the surface of a commercially available standard endotracheal tube (Unomedical Air Management, Magill, HVLP cuff) to yield four square-shaped electrodes situated on the surface of the cuff. The electrodes were extended with a narrower written trace down the tube for a length of approximately 10 cm to allow for subsequent interconnection with external devices. The ink was cured by forced air, at 130° C., for 1 hour.

After the silver pads and leads were cured, they were covered, except for small connection pads at the end of each lead and the center of each square-shaped electrode, with a UV-curable medical polymeric encapsulant (Dymax 1-20323; Dymax Corporation). The medical polymeric encapsulant was subsequently cured via ultraviolet irradiation.

For production of the reference electrode, polyvinyl chloride (Aldrich, high molecular weight) was dissolved in a combination of tetrahydrofuran/N-methylpyrrolidone (60/40 ratio) at a concentration of 1% by weight. Silver flakes (Aldrich, <10 microns) and silver chloride powder (Silver (I) Chloride, 99.9% metal basis, Alfa Aesar; lightly crushed to reduce agglomerate size) were added in a ratio of 3:1 to each other; and 1.33:1 to the polyvinyl chloride binder polymer, yielding an ink which had 25% solids by weight.

The reference electrode ink was loaded into a syringe and dispensed onto the center of a square-shaped electrode situated on an endotracheal tube cuff as described above. The ink was cured at 110° C. for 30 minutes, then the reference electrode ink was re-applied in order to cover any cracks and cured again under the same conditions. A layer of Nafion® perfluorinated resin solution (5 wt. % in mixture of lower aliphatic alcohols and water, contains 45% water (Aldrich)) was deposited over the reference electrode and cured at 50° C. for 30 minutes.

For production of the working electrode, polyvinyl chloride (Aldrich, High molecular weight) was dissolved in a combination of tetrahydrofuran/N-methylpyrrolidone (60/40 ratio) at a concentration of 1% by weight. Ruthenium oxide (Ruthenium(IV) oxide, 99.9% trace metals basis, Aldrich) was added such that the ratio of RuO₂ to polyvinyl chloride was 90:10 and the total solids present in the ink were 9% by weight.

This active electrode ink was loaded into a syringe and dispensed onto the center of a square-shaped electrode situated on an endotracheal tube cuff as described above. The ink was cured at 110° C. for 30 minutes. The reference electrode ink was the re-applied in order to cover any cracks and cured again under the same conditions.

To test the system, leads were attached to the electrodes bearing the reference and active electrode inks, and the entire cuff was immersed in pH buffer reference standards (pH 4, 7 and 10; Sigma). For each buffer standard, voltage was read and recorded. Between each immersion, the cuff was thoroughly rinsed in deionized water.

The results are illustrated in Table 1, indicating that the electrochemical sensor accurately measures hydrogen ion concentration, which is reflected in the pH value.

TABLE 1
pH Buffer Reference Standards-pH and Voltage (mV)

| Buffer pH | mV |
|---|---|
| 4 | 124 |
| 7 | −31 |
| 10 | −142 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A device for sensing a target chemical comprising:
a flexible, non-planar substrate;
a printed, solid-state sensing element comprising a chemical sensing material which produces an electrical signal upon interaction with the target chemical;
a first printed electrode comprising a first conductive composition; and
a second electrode comprising a second conductive composition, wherein said first and second electrodes are electrically isolated from one another, and one or both of the first and second electrodes is in electrical contact with said sensing element, wherein said first and second electrodes and said sensing element collectively form an electrochemical sensor, which is coupled to said flexible, non-planar substrate.

2. The device according to claim 1, wherein the first printed electrode is in electrical contact with said sensing element.

3. The device according to claim 1, wherein both the first and second electrodes are in electrical contact with said sensing element.

4. The device according to claim 1, wherein the second electrode is printed onto the substrate.

5. The device according to claim 1, wherein the non-planar substrate has an irregular surface.

6. The device according to claim 1, wherein the first and second electrodes are independently formed from a material selected from the group consisting of copper; silver; gold; palladium; platinum; nickel; graphite; carbon black; conductive carbon; conductive ceramics; tin oxide; vanadium pentoxide; doped versions of tin oxide; doped versions of vanadium oxide; conductive polymers of polypyrrole, polythiophene, polyaniline, and mixtures or copolymers thereof.

7. The device according to claim 1, wherein the chemical sensing material is selected from the group consisting of an ionophore, an enzyme, a macromolecule, a metal, a metal oxide or a metal nitride, an insertion compound which physically entraps target species through geometrical action, cyclodextrin, zeolite, or other material or combinations thereof.

8. The device according to claim 1, wherein the first or second electrode is a reference electrode.

9. The device according to claim 1 further comprising an electrical measurement device coupled to the first and/or the second electrode.

10. The device according to claim 9, wherein the electrical measurement device is selected from the group consisting of a voltmeter, ohmmeter, oscilloscope, and ammeter.

11. The device according to claim 1 further comprising:
an overcoat layer at least partially coating the electrochemical sensor.

12. The device according to claim 11, wherein the overcoat layer is formed from a material selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), ionomers, and mixtures or copolymers thereof.

13. The device according to claim 1 further comprising:
an intermediate layer positioned between the flexible, non-planar substrate and one or more of the first printed electrode, the second electrode, and the sensing element.

14. The device according to claim 13, wherein the intermediate layer is formed from a material selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), and mixtures or copolymers thereof.

15. The device according to claim 1, wherein the first and second conductive compositions comprise a binder selected from the group consisting of poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polyethylene, polytetrafluoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polyisoprene, polycaprolactone, cyanoacrylates, polyvinyl butyral, polyvinyl formal, polyethylene oxide, polyvinyl alcohol, polyvinylpyrrolidone, cellulose esters, cellulose ethers, carrageenan, gelatin, chitosan, and mixtures or copolymers thereof.

16. The device according to claim 1, wherein the substrate is formed from a material selected from the group consisting of polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafluoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polycaprolactone, and copolymers or mixtures thereof.

17. The device according to claim 1, wherein the electrochemical sensor has a thickness of 1 to 500 microns.

18. The device according to claim 1, wherein the electrochemical sensor has a thickness of 20 to 100 microns.

19. The device according to claim 1, wherein said device comprises a plurality of said electrochemical sensors.

20. A medical device comprising the device according to claim 1.

21. The medical device according to claim 20, wherein said medical device comprises a plurality of said electrochemical sensors.

22. The medical device according to claim 20, wherein the medical device is selected from the group consisting of an endotracheal tube, endobronchial tube, cannula, catheter, balloon, stent, airway, sensor, stimulator, implant, intraocular or contact lens, cochlear implant, and orthopedic implant or prosthesis.

23. A method of forming a device for sensing a target chemical, the method comprising:
providing a flexible, non-planar substrate; and
printing an electrochemical sensor on said flexible, non-planar substrate, said electrochemical sensor comprising:
a first electrode comprising a first conductive composition and
a solid-state sensing element comprising a chemical sensing material which produces an electrical signal upon interaction with the target chemical, wherein the sensing element is electrically coupled to the first electrode.

24. The method according to claim 23, wherein the electrochemical sensor further comprises a second electrode electrically isolated from the first electrode, said second electrode comprising a second conductive composition.

25. The method according to claim 24, wherein both the first and second electrodes are in electrical contact with the sensing element.

26. The method according to claim 24, wherein the second electrode is printed onto the substrate.

27. The method according to claim 24, wherein the first and second electrodes are independently formed from a material selected from the group consisting of copper; silver; gold; palladium; platinum; nickel; graphite; carbon black; conductive carbon; conductive ceramics; tin oxide; vanadium pentoxide; doped versions of tin oxide; doped versions of vanadium oxide; conductive polymers of polypyrrole, polythiophene, polyaniline, and mixtures or copolymers thereof.

28. The method according to claim 24, wherein the first or second electrode is a reference electrode.

29. The method according to claim 24 further comprising:
applying an intermediate layer between the non-planar substrate and one or more of the first electrode, the second electrode, and the sensing element.

30. The method according to claim 24, wherein the first and second conductive compositions comprise a binder selected from the group consisting of poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polyethylene, polytetrafluoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polyisoprene, polycaprolactone, cyanoacrylates, polyvinyl butyral, polyvinyl formal, polyethylene oxide, polyvinyl alcohol, polyvinylpyrrolidone, cellulose esters, cellulose ethers, carrageenan, gelatin, chitosan, and mixtures or copolymers thereof.

31. The method according to claim 23, wherein the flexible, non-planar substrate has an irregular surface.

32. The method according to claim 23, wherein the chemical sensing material is selected from the group consisting of an ionophore, an enzyme, an enzyme substrate, a macromolecule, a metal, a metal oxide or a metal nitride, an insertion compound which physically entraps target species through geometrical action, cyclodextrin, zeolite, or other material or combinations thereof.

33. The method according to claim 23 further comprising:
applying an overcoat layer at least partially coating the electrochemical sensor.

34. The method according to claim 23, wherein the overcoat layer is formed from a material selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), ionomers, and mixtures or copolymers thereof.

35. The method according to claim 23, wherein the intermediate layer is formed from a material selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), and mixtures or copolymers thereof.

36. The method according to claim 23, wherein the substrate is formed from a material selected from the group consisting of polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactide, polyglycolide, polycaprolactone, and copolymers or mixtures thereof.

37. The method according to claim 23, wherein the electrochemical sensor has a thickness of 1 to 500 microns.

38. The method according to claim 23, wherein the electrochemical sensor has a thickness of 20 to 100 microns.

39. The method according to claim 23, wherein said printing the electrochemical sensor is carried out by direct writing.
